(19) United States
(12) Patent Application Publication
(10) Pub. No.: US 2021/0346065 A1
(43) Pub. Date: Nov. 11, 2021

(54) SPINAL SCREW

(71) Applicant: GLOBUS MEDICAL, INC., Audubon, PA (US)

(72) Inventors: Stefan P. Lamers, Phoenixville, PA (US); Matthew Bechtel, Philadelphia, PA (US); Jeshurun S. Paul, Phoenixville, PA (US); David C. Paul, Phoenixville, PA (US)

(21) Appl. No.: 17/226,171

(22) Filed: Apr. 9, 2021

Related U.S. Application Data
(60) Provisional application No. 63/007,429, filed on Apr. 9, 2020.

(51) Int. Cl. A61B 17/70; A61B 17/86; A61B 34/20; A61B 34/30; A61B 90/00

(52) U.S. Cl. CPC A61B 17/7082; A61B 17/864; A61B 34/20; A61B 2090/031; A61B 90/39; A61B 90/03; A61B 2034/2046; A61B 34/30

(57) ABSTRACT

A spinal screw for positioning within bone for use in surgical procedures, the spinal screw having a head and a shaft, the shaft having a proximal end and a distal end. A through-hole extends from the head through the shaft to the distal end of the shaft. The through-hole defines a longitudinal axis of the spinal screw and a spring is positioned within the shaft. A retractable tip element is also positioned within the through hole of the shaft and is operationally coupled to the spring.

### CROSS-REFERENCE TO RELATED APPLICATIONS

This application is a non-provisional application that claims priority to Provisional Application 63/007,429 filed on Apr. 9, 2020, which is incorporated in its entirety herein.

### FIELD OF THE INVENTION

The present invention relates to a system for stabilizing the spine, and more particularly to a spinal screw that reduces the risk of bone fracturing and reduces spinal screw insertion torque.

### BACKGROUND OF THE INVENTION

The human spine consists of individual vertebras that are connected to each other. Under normal circumstances, the structures that make up the spine function to protect the neural structures and to allow us to stand erect, bear axial loads, and be flexible for bending and rotation. However, disorders of the spine occur when one or more of these spine structures are abnormal. In these pathologic circumstances, surgery may be tried to restore the spine to normal, achieve stability, protect the neural structures, or to relieve the patient of discomfort. The goal of spine surgery for a multitude of spinal disorders, especially those causing compression of the neural structures, is often decompression of the neural elements and/or fusion of adjacent vertebral segments. Fusion works well because it stops pain due to movement at the facet joints or intervertebral discs, holds the spine in place after correcting deformity, and prevents instability and or deformity of the spine after surgical procedures such as discectomies, laminectomies, or corpectomies.

Several spinal fixation systems exist for stabilizing the spine so that bony fusion is achieved. The majority of these fixation systems utilize fixation elements such as rods, wires, or plates that attach to screws threaded into the vertebral bodies, facets, or the pedicles. Because the outer surface of the vertebral body is typically non-planar and the structure of the vertebras is relatively complex, it is important that the fixation elements (e.g., rods, plates, wires, staples and/or screws) are properly aligned when they are inserted into the vertebras. Improper alignment may result in improper or unstable placement of the fixation element and/or disengagement of the fixation element.

Achieving and maintaining accurate positioning and guidance of these fixation elements, however have proven to be quite difficult in practice. Such positioning difficulties are further complicated by the fact that the alignment angle for a fixation device through one vertebral body or pair of vertebral bodies will be unique to that individual due to individual differences in the spinal curvature and anatomies. Accordingly, there is a need for a spinal screw assembly that reduces insertion torque of a self-drilling screw design without compromising fixation attributes. Thus, there is a need for a self-drilling spinal screw that reduces the risk of bone fracturing during insertion, reduce the need for drilling and tapping, and reduce overall operating room time.

### SUMMARY OF THE INVENTION

The disclosure meets the foregoing need and uses spinal screws to reduce insertion torque and risk of bone fractures during the placement of the spinal screws into bone.

Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

A spinal screw for positioning within bone for use in surgical procedures, the spinal screw having a head and a shaft, the shaft having a proximal end and a distal end. A through-hole extends from the head through the shaft to the distal end of the shaft. The through-hole defines a longitudinal axis of the spinal screw and a spring is positioned within the shaft. A retractable tip element is also positioned within the through hole of the shaft and is operationally coupled to the spring.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 is perspective view of a spinal screw in accordance with exemplary embodiments of the present disclosure;

FIG. 2 illustrates the spinal screw of FIG. 1 in accordance with exemplary embodiments of the present disclosure .

FIG. 3 is a perspective view of a spinal screw in accordance with exemplary embodiments of the present disclosure;

FIGS. 4 and 5 are views of the distal end of a spinal screw in accordance with exemplary embodiments of the present disclosure;

FIGS. 6 and 7 are view of the distal end of a spinal screw in accordance with exemplary embodiments of the present disclosure;

FIGS. 8 and 9 illustrate yet another embodiment of a spinal screw in accordance with exemplary embodiments of the present disclosure;

FIGS. 10 and 11 illustrate a spinal screw in accordance with exemplary embodiments of the present disclosure; and

### DETAILED DESCRIPTION OF THE INVENTION

It is to be understood that the present disclosure is not limited in its application to the details of construction and the arrangement of components set forth in the description herein or illustrated in the drawings. The teachings of the present disclosure may be used and practiced in other embodiments and practiced or carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the present disclosure. Various modifications to the illustrated embodiments will be readily apparent to those skilled in the art, and the principles herein can be applied to other embodiments and applications without departing from embodiments of the present disclosure. Thus, the embodiments are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the embodiments. Skilled artisans will recognize the examples provided herein have many useful alternatives and fall within the scope of the embodiments.

Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments or modifications thereof are contemplated as falling within the scope of this disclosure and equivalents thereto.

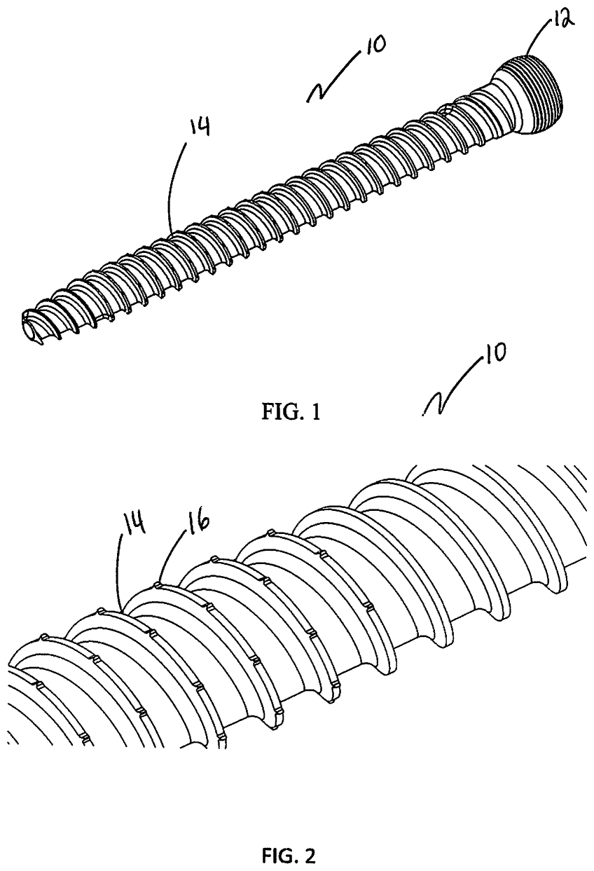

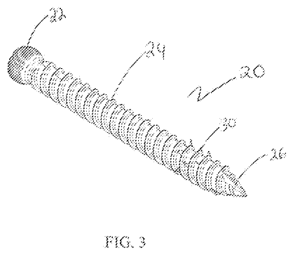

FIGS. 1 and 2 illustrate a spinal screw 10 configured to be used in surgical procedures. The spinal screw 20 comprises a head portion 12 and shaft portion 14. The shaft portion 14 is provided with threading to enable the spinal screw 10 to be rotated into bone. The shaft portion 14 in this particular embodiment is provided with protrusions 16 extending from the threads of the shaft portion 14, as more clearly shown in FIG. 2. The protrusions 16 provide an external cutting peak that is slightly over the major diameter of the spinal screw 10. The protrusions 16 allow for the decrease in the insertion torque of the spinal screw 10 while effectively eliminating the need to drill and tap within the bone prior to the spinal screw 10 insertion. The spinal screw 10 as illustrated is provided with a self-tapping thread profile and a pitch that enables the spinal screw 10 to be a self-drilling and tapping screw. It should be noted the spinal screw 10 may be configured in various lengths, and diameters. Also, the protrusions 16 may be modified in various shapes such as hooks, pyramids, rounded edges, and any configuration that allows for the decrease in the insertion torque of the spinal screw 10.

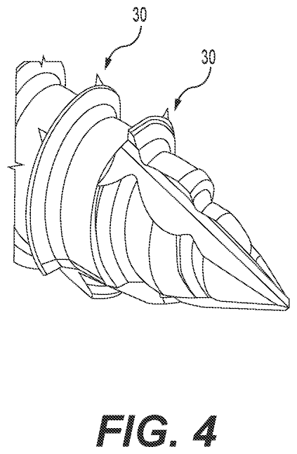

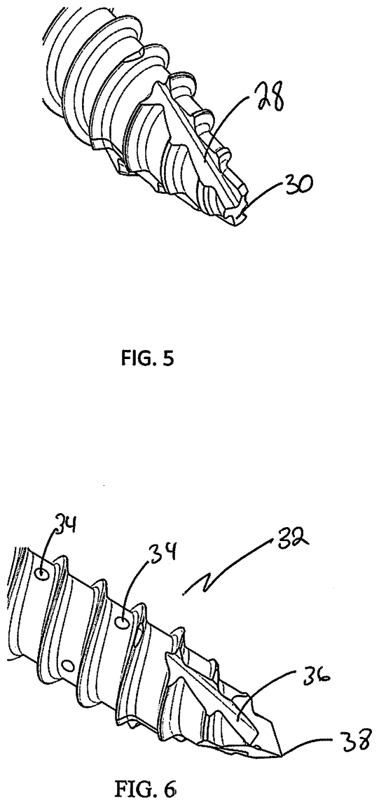

FIG. 3 illustrates yet another spinal screw 20 with a head portion 22 and a shaft portion 24. The distal end of the shaft portion 24 is configured with a cutting flute 26 and configured to cut bone. FIG. 4 illustrates a closer view of the cutting flute 26 in accordance with an exemplary embodiment of the present invention. The distal end of the shaft portion 24 in this embodiment provides a cutting flute 26 with a sharp tip. In another embodiment the distal end of the shaft portion 24 of a spinal screw is provided with a cutting flute 28 with blunt flat end 30 as shown in FIG. 5. The cutting flutes shown in FIGS. 4 and 5 are configured to decrease the insertion torque of the spinal screw while effectively eliminating the need to drill and tap prior to the spinal screw insertion into bone. The spinal screws of FIGS. 4 and 5 may also be provided with protrusions 32 to decrease the insertion torque required for insertion of the spinal screw into bone. These protrusions may be configured to extend the entire length of the shaft portion of the spinal screw or localized to different sections of the shaft portion of the spinal screw.

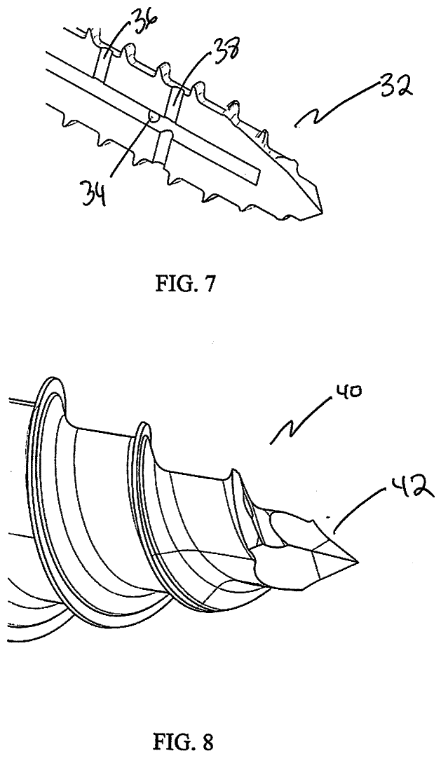

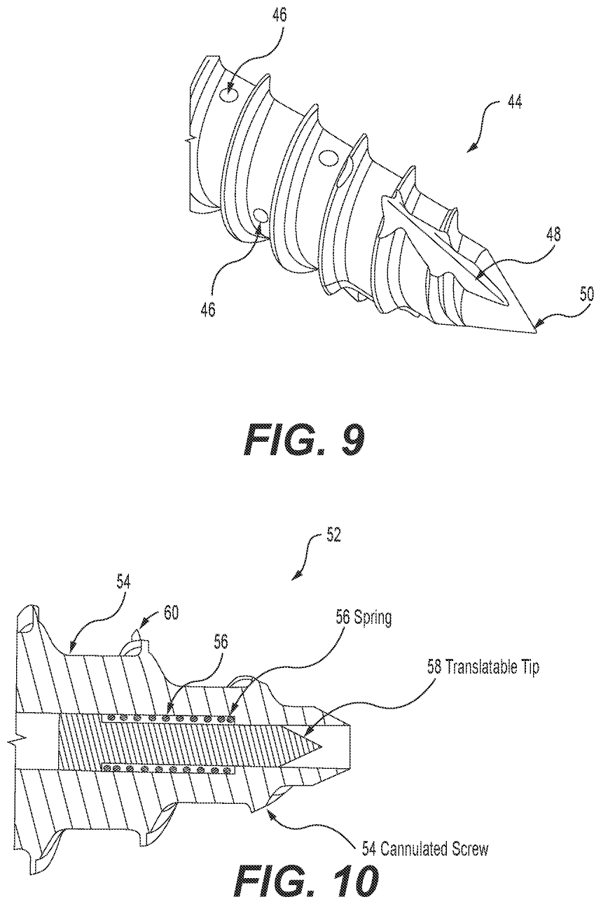

In another embodiment, as shown in FIG. 6. a spinal screw 32 is shown having fenestrations 34, a cutting flute 36, and a sharp tip 38. FIG. 7 illustrates a cross sectional view of the spinal screw 32 illustrating the fenestrations 34. The fenestrations as shown can be configured as blind holes 36 or through holes 38 extending through the shaft of the spinal screw 32. In another embodiment, FIG. 8 illustrates a spinal screw 40 that includes a threaded trocar tip 42 and FIG. 9 illustrates the spinal screw 44 with fenestrations 46, a cutting flute 48 and the threaded trocar tip 50. It should be noted that the spinal screw shown in all the above embodiments may also be cannulated. The fenestration and the cannulations provided in the screw enable the insertion of bone cement, autograft, allograft or any combination thereof.

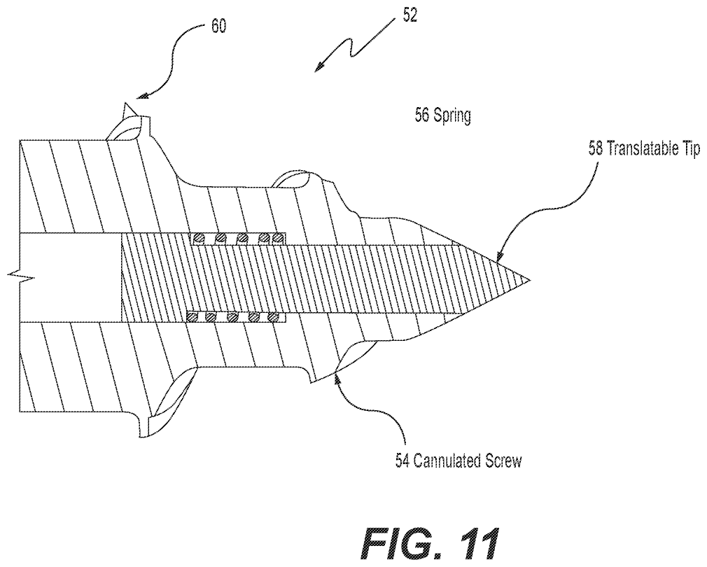

Now, turning to FIGS. 10 and 11, a multi-piece spinal screw 52 is provided . Spinal screw 52 includes a cannulated screw portion 54, an internal spring 56, and internal translatable tip 58 centered within the cannulated screw 54. The cannulated screw 54 is provide with a head portion and a shaft portion, the shaft portion may be configured with or without a cutting flute. The cannulated screw 54 may also be configured with or without protrusions 60. The internal spring 56 is positioned within a groove on an inner shelf at the distal end of the cannulated screw 54. The translatable tip 58 is positioned within the cannulation of the screw and configured to protrude out the distal end of the cannulated screw as the driver is attached and pushes on the back end of the translatable tip 58. The translatable tip 58 will retract into the cannulated screw 54 after the back of the tip is disengaged. The multi-piece spinal screw 52 as shown in FIGS. 10 and 11 improves the efficiency and the speed at which the bone screw is inserted during surgery. The exemplary embodiment of the spinal screw 52 allows for the better placement of the screw within bone, reduces surgical time, provides a reduction in x-ray exposure, and a reduction in anesthesia exposure over traditional bone screws. The spinal screw according to the present disclosure also reduces the force exerted on the bone reducing the potential cracks and necrosis of the bone.

In an alternative embodiment, the multipiece retractable tip screw may utilize alternative means for extending and retracting the tip. For example, a ratchet type mechanism may be utilized to extend the tip into bone and retracted by release of the ratchet mechanism. In another embodiments, the tip may be extended various lengths depending on the need of the patient.

The spinal screw as provide in FIGS. 1-11 are configured to be utilized in robotic applications. The spinal screw is configured to be trackable by a robotic system that includes a camera system, and a robotic arm coupled to a robot base. The robotic system includes a computer system that is configured to navigate the spinal screw using trackable markers that are visible to the camera system. The navigation system used by the robotic system also is capable of registering images received by an imaging device such a CT, MRI or fluoroscope to the images received by the camera system. The spinal screw is also configured to attach to trackable markers mechanically and electrically. In another embodiment the spinal screw is provided with bone sensors to determine the density of the bone. In some embodiments, sensors may be positioned on the spine screw to determine bone density and other bone characteristics. In one embodiment, as the spinal screw is threaded into bone, the retractable tip element is retracted into the spinal screw if the sensor no longer senses bone or a reduced bone density.

One skilled in the art will appreciate that the embodiments discussed above are non-limiting. While devices may be described as suitable for a particular location (e.g., vertebra) or approach, one skilled in the art will appreciate that the devices, instruments, and methods described herein can be used for multiple locations and approaches. In addition to the devices, instruments, and methods described above, one skilled in the art will appreciate that these described features can be used with a number of other implants and instruments, including fixation plates, rods, fasteners, and other orthopedic devices. It will also be appreciated that one or more features of one embodiment may be partially or fully incorporated into one or more other embodiments described herein.

What is claimed is:

1. A spinal screw for positioning within bone for use in surgical procedures, the spinal screw comprising:
a head and a shaft, the shaft having a proximal end and a distal end;
a through-hole extending from the head through the shaft to the distal end of the shaft; the through-hole defining a longitudinal axis of the spinal screw;
a spring positioned within the shaft; and
a retractable tip element positioned within the through hole of the shaft and operationally coupled to the spring;
wherein the retractable tip element includes a first end and a second end, the first end capable of receiving an instrument engaging with the retractable tip, the second end having a tip that engages with bone,
wherein when pressure is applied to the instrument, the retractable tip is engaged and pushed to extend the tip of the retractable tip element past the distal end of the shaft to engage with bone,
wherein when pressure on the instrument is no longer applied, the retractable tip element is disengaged from the bone,

2. The spinal screw of claim 1, wherein the shaft of the spinal screw includes threads extending from along the length of the shaft.

3. The spinal screw of claim 2, wherein a plurality of protrusions are provided on a major diameter of the spinal screw.

4. The spinal screw of claim 1, wherein the shaft includes a plurality of fenestrations.

5. The spinal screw of claim 4, wherein the fenestrations include blind holes.

6. The spinal screw of claim 4, wherein the fenestrations include through holes.

7. The spinal screw of claim 1, wherein the spinal screw is threaded into bone and the retractable tip element is retracted in the spinal screw when a predetermined depth is reached.

8. The spinal screw of claim 1, wherein the spinal screw is threaded into bone and the retractable tip element is retracted in the spinal screw when no pressure on the retractable tip is sensed by a bone sensor positioned on the distal tip of the retractable tip element.

9. The spinal screw of claim 1, wherein the spinal screw includes a plurality of tracking markers for use for with a robotic surgical system.

10. A spinal screw for positioning within bone for use in surgical procedures, the spinal screw comprising:
a head and a shaft, the shaft having a proximal end and a distal end;
a through-hole extending from the head through the shaft to the distal end of the shaft; the through-hole defining a longitudinal axis of the spinal screw;
a spring positioned within the shaft; and
a retractable tip element positioned within the through hole of the shaft and operationally coupled to the spring.

11. The spinal screw of claim 10, wherein the shaft of the spinal screw includes threads extending from along the length of the shaft.

12. The spinal screw of claim 11, wherein a plurality of protrusions are provided on a major diameter of the spinal screw.

13. The spinal screw of claim 10, wherein the shaft includes a plurality of fenestrations.

14. The spinal screw of claim 13, wherein the fenestrations include blind holes.

15. The spinal screw of claim 13, wherein the fenestrations include through holes.

16. The spinal screw of claim 10, wherein the spinal screw is threaded into bone and the retractable tip element is retracted in the spinal screw when a predetermined depth is reached.

17. The spinal screw of claim 10, wherein the spinal screw is threaded into bone and the retractable tip element is retracted into the spinal screw when no pressure on the retractable tip is sensed by a bone sensor positioned on the distal tip of the retractable tip element.

18. The spinal screw of claim 10, wherein the spinal screw includes a plurality of tracking markers for use for with a robotic surgical system.

19. The spinal screw of claim 10, wherein the retractable tip element includes a first end and a second end, the first end capable of receiving an instrument engaging with the retractable tip, the second end having a tip that engages with bone,
wherein when pressure is applied to the instrument, the retractable tip is engaged and pushed to extend the tip of the retractable tip element past the distal end of the shaft to engage with bone,
wherein when pressure on the instrument is no longer applied, the retractable tip element is disengaged from the bone,

20. The spinal screw of claim 13, wherein the protrusions are configured to reduce insertion torque.
